(19) United States
(12) Patent Application Publication
(10) Pub. No.: US 2002/0010417 A1
(43) Pub. Date: Jan. 24, 2002

(54) Transpharyngeal tube

(76) Inventor: Volker Bertram, Sulz (DE)

Correspondence Address: Eugene LeDonne, Esq., Reed Smith LLP, 17th Floor, 375 Park Ave., New York, NY, 10152, US

(21) Appl. No.: 09/741,965

(22) Filed: Dec. 21, 2000

(30) Foreign Application Priority Data
Dec. 24, 1999 (DE) DE 199 62 985.4

(51) Int. Cl. A61M 29/00

(52) U.S. Cl. 604/96.01

(57) ABSTRACT

A transpharyngeal tube for intubation anaesthesia which includes an inherently stable tube shaft with an inflatable pharyngeal cuff for blocking the pharynx and an inflatable esophageal cuff for blocking the esophagus. One shaft end of the tube shaft facing the esophagus is provided with a tube tip of an easily deformable material which is softer than that of the tube shaft. Strain of the patient when contacting an inner wall of the esophagus with the tube tip has been further reduced.

### RELATED PATENTS AND APPLICATIONS

This application claims Paris Convention priority of DE 199 62 985.4 filed on Dec. 24, 1999, the complete disclosure of which is hereby incorporated by reference.

### FIELD OF THE INVENTION

The invention relates to a transpharyngeal tube and, more particularly, to a transpharyngeal tube for intubation anaesthesia including an inherently stable tube shaft with an inflatable pharyngeal cuff for blocking the pharynx and an inflatable esophageal cuff for blocking the esophagus.

### BACKGROUND OF THE INVENTION

A transpharyngeal tube of this type is disclosed e.g. in DE 195 37 735 C. A transpharyngeal tube is an alternative to artificial respiration by means of a so-called larynx mask or also to endotracheal tubes if there is no danger of aspiration. Due to its simplicity, the transpharyngeal tube is an ideal supplement for emergency artificial respiration.

A possible application of intubation anaesthesia includes, in most cases after premedication of the patient, initial intravenous anaesthesia or after inhalation anaesthesia, introduction of the transpharyngeal tube into the patient. If the tip of the tube is introduced only to the entrance region of the esophagus, and the esophageal cuff blocks only said entrance region, painful strain of the esophagus is prevented. The activated esophageal cuff occludes the entrance to the esophagus in a gas and liquid-tight manner.

When the transpharyngeal tube has been introduced, the trachea is sealed off by blocking the cuff in the esophagus and in the pharynx to provide at any time a free respiratory path for the patient, separate from the esophagus, by means of an application channel of the tube, and to prevent in particular any mixing of vomitted stomach contents and respiratory air. Artificial respiration of the patient is now possible via the application channel in the inside of the transpharyngeal tube. It is possible to add an inhalation anaesthesia by supplying anaesthetic, e.g. an air or O₂ anaesthetic mixture.

The tube shaft is usually produced from an inherently stable plastic material, such as silicone. For this reason, the tube tip has to be carefully introduced into the esophagus entrance to avoid unnecessary strain of the patient. Contact of the tube tip with the inner wall of the pharynx, however, produces unavoidable strain.

It is the underlying purpose of the present invention to further reduce the strain of the patient when the tube tip contacts an inner wall of the pharynx.

### SUMMARY OF THE INVENTION

This object is achieved in accordance with the invention by a transpharyngeal tube having an inherently stable tube shaft whose shaft end facing the esophagus comprises a tube tip made from a deformable material which is softer than the tube shaft. The tube tip is produced from a material which easily yields under pressure and can be deformed and compressed. The tube tip can change its shape under little force and subsequently assume its original shape again. For this reason, the tube tip can adapt well to the contours of a hollow organ and fit smoothly to the inner wall of the hollow organ.

In one embodiment of the invention, the tube tip is produced of foamed material or expanded rubber or of another soft material, such as soft silicone and inserted into a shaft end of the silicone tube shaft, to obtain a resilient tip having good guiding properties of the tube shaft. In the ideal case, a tube tip of soft silicone has a hardness of approximately 20 Shore A and the tube shaft a hardness of approximately 60 Shore A. Due to the natural stability of the tube shaft, the transpharyngeal tube can be introduced directly to the esophagus entrance. The soft tube tip will not injure or strain the patient despite contacting him/her. The porous structure of the foamed material as well as its softness ideally provide its resilience which shows already under slight contact.

It is particularly preferred that the esophageal cuff surrounds the tube tip on all sides. The esophageal cuff may serve as collecting means for a tube tip in case the tube tip unexpectedly becomes detached from the tube shaft.

The tube tip may have different geometric outer contours. The tube tip of one embodiment has a wedge-shaped tip body which changes into a mounting body for insertion into the shat end. The wedge shape provides good support on the vessel wall of the esophagus entrance.

In a further development of the invention, the tip body is provided with an abutment surface for limited use of the tube tip which prevents the tube tip from sliding into the tube shaft or penetrating into the esophagus.

One embodiment of the inventive transpharyngeal tube is shown in the schematic drawing and is explained in the following description with respect to the drawing.

### BRIEF DESCRIPTION OF THE DRAWING

FIG. 1 shows the entrance region of the esophagus with inserted transpharyngeal tube;

FIG. 2a shows a side view of a tube tip for the transpharyngeal tube in accordance with FIG. 1;

FIG. 2b shows an enlarged partial area of the patient-side tube end of the transpharyngeal tube in accordance with FIG. 1;

FIG. 2c shows an enlarged partial area of another patient-side tube end;

FIG. 2d shows an enlarged partial area of another patient-side tube end.

### BRIEF DESCRIPTION OF THE PREFERRED EMBODIMENT

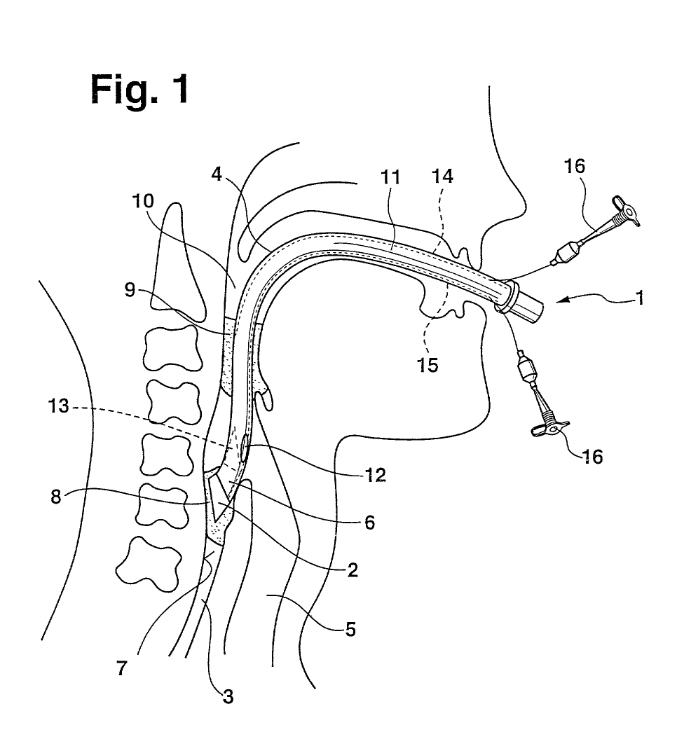

FIG. 1 shows the application of a transpharyngeal tube 1 whose tube tip 2 has been introduced into the entrance region of the esophagus 3. The transpharyngeal tube 1 comprises an essentially inherently stable tube shaft 4 of silicone or another plastic material having an S-shaped longitudinal profile or being at least straightened along the length of the two cuffs. The longitudinal profile formed in this fashion, guarantees that the transpharyngeal tube 1 is always introduced into the entrance region of the esophagus 3 and not wrongly into the trachea 5.

Due to the S-shape of the tube shaft 4 made from a flexible elastic plastic material, the tube tip 2 rests with slight pressure on the inner wall 6 of the esophagus 3 due to the spring effect of the tube shaft 4 thereby fixing the tube tip 2 in the entrance region of the esophagus 3. To maintain the tube tip 2 in this position, an esophageal cuff 8 is ventilated blocking the entrance region of the esophagus 3 in a gas and liquid-tight manner. When the transpharyngeal tube 1 is introduced, the esophageal cuff 8 is folded and put into a cavity of the tube tip 2 to ensure easy insertion of the transpharyngeal tube 1. The esophageal cuff 8 may be disposed at the tube tip 2 in such a fashion that it does not increase the outer diameter of the tube tip 2.

The pharynx 10 is proximally blocked via an inflatable pharyngeal cuff 9. The esophagus is distally blocked by the esophageal cuff 8. This produces and end-to-end connection between the trachea (larynx) 5 as natural respiratory path and the transpharyngeal tube 1 via an application channel 11 without reducing the lumen of the transpharyngeal tube 1 with respect to the trachea 5. On the one hand, the treated patient is artificially supplied with air via the application channel 11 and on the other hand anaesthesized by supplied application means, such as anaesthesia. Application means may flow into the trachea 5 via the application channel 11 and an application opening 12. The application channel 11 is continuously formed in the tube shaft 4 and is subdivided by a blocking element 13 into an upper part used for application and the cavity of the tube tip 2. The blocking element 13 forms a flow aid which may guide in flowing application means, a catheter or fiber bronchoscope to the application opening 12.

The pharyngeal cuff 9 and the esophageal cuff 8 may be ventilated and inflated or deaerated in a controlled manner with adjustable inner pressure in each case via a ventilation channel 14 or 15 formed in the shaft wall. The ventilation channel 14 extends from a blocking device 16 to the pharyngeal cuff 9. The ventilation channel 15 extends from the blocking device 16 to the esophageal cuff 8. Known means for inflating a cuff 8 and 9 and common pressure manometers may be attached to the blocking device 15.

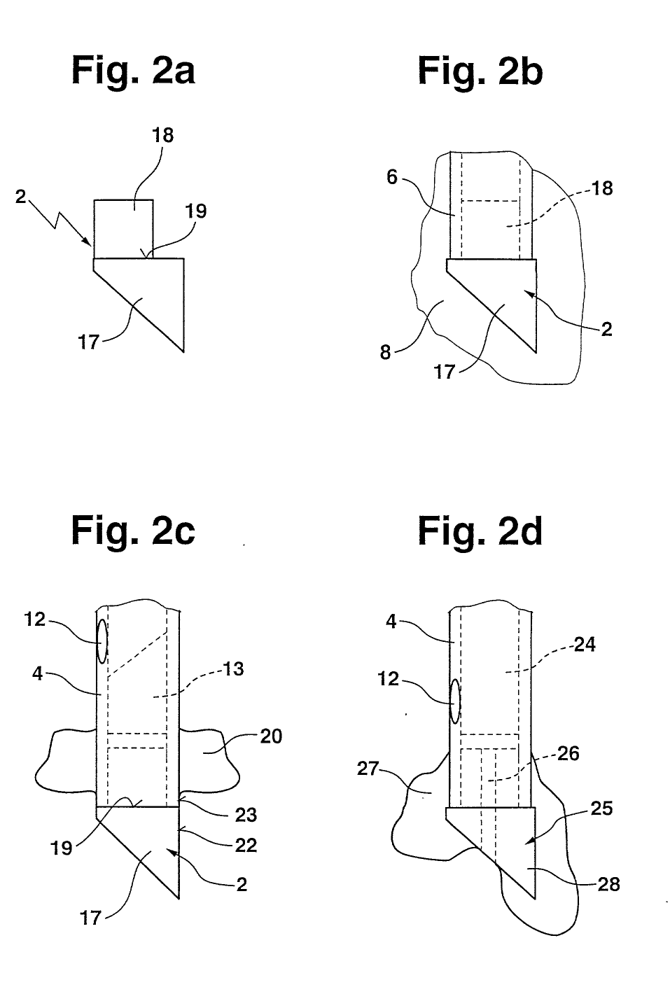

FIG. 2a shows a side view of a tube tip 2. The tube tip 2 comprises a tip body 17 which projects into the esophagus cuff 8 and is surrounded by same from all sides. The tip body 17 changes step by step into a mounting body 18 which can be fixed in the shaft end of the tube shaft. The tube tip 2 can be inserted into the shaft end to a continuous abutment surface 19. FIG. 2b shows clearly the connection of the tube tip 2 of elastic foamed material or another soft material, which is essentially softer than the tube shaft 4, with the silicone shaft end 6. The mounting body 18 is inserted into the shaft end 6 and glued to same. Tube tips 2 of different geometrical shapes can be inserted into the shaft end 6.

FIG. 2c shows an alternative embodiment of a tube end facing the esophagus. The tube tip 2 comprises the tip body 17 which forms the tip of a transpharyngeal tube. A cylindrical esophageal cuff 20 is mounted to the tube shaft 4 and surrounds the tube shaft 4 from the side, wherein an abutment surface 19 of the tip body 17 covers the distal glue surface of the esophageal cuff 20. The outer surface 22 of the tip body 17 and the outer surface 23 of the esophageal cuff 20 gradually merge. FIG. 2c further shows the blocking element 13 and the application opening 12.

The patient-side end of the transpharyngeal tube may also be designed such that no blocking element is provided in the lumen 24 of the tube shaft 4 as shown in FIG. 2d. A tube tip 25 comprises a lumen 26 to provide e.g. insertion of a suction catheter into the esophagus. The laterally and distally disposed esophageal cuff 27 surrounds the lumen 26 of a tip body 28 in the direction of the esophagus in the ventilated state. The ventilated esophageal cuff 27 may also sealingly surround a catheter inserted into the tip body 28 and/or projecting beyond the lumen 26.

I claim:

1. A transpharyngeal tube for intubation anaesthetic, said tube comprising:
an inherently stable tube shaft with an inflatable pharyngeal cuff for blocking the pharynx; and,
an inflatable esophageal cuff for blocking the esophagus;
wherein one shaft end of the tube shaft facing the esophagus is provided with a tube tip of an easily deformable material which is softer than that of the tube shaft.

2. The transpharyngeal tube in accordance with claim 1, wherein a tube tip of foamed material is inserted into a shaft end of a tube shaft of silicone.

3. The transpharyngeal tube in accordance with claim 1, wherein the esophageal cuff surrounds the tube tip on all sides.

4. The transpharyngeal tube in accordance with claim 1, wherein the tube tip comprises a wedge-shaped tip body which merges into an attachment body for insertion into the shaft end.

5. The transpharyngeal tube in accordance with claim 4, wherein the tip body is provided with an abutment surface for limited insertion of the tube tip.
